(19) United States
(12) Patent Application Publication
(10) Pub. No.: US 2010/0292609 A1
(43) Pub. Date: Nov. 18, 2010

(54) Method for manufacturing a carrier tape comprising lancets and tape magazines for storing lancets

(76) Inventors: Volker Zimmer, Morbach (DE); Frank Deck, Niederkirchen (DE); Bernd Hiller, Lampertheim (DE); Ahmet Konya, Waldsee (DE)

Correspondence Address: ROCHE DIAGNOSTICS OPERATIONS INC., 9115 Hague Road, Indianapolis, IN, 46250-0457, US

(21) Appl. No.: 12/619,406

(22) Filed: Nov. 16, 2009

Related U.S. Application Data
(63) Continuation of application No. PCT/EP2008/055689, filed on May 8, 2008.

(30) Foreign Application Priority Data
May 15, 2007 (EP) 07108252.3

(51) Int. Cl. A61B 5/151; B23P 11/00; B65B 35/30

(52) U.S. Cl. 600/583; 29/428; 53/448

(57) ABSTRACT

A method for manufacturing a carrier tape comprising lancets and corresponding products in which a plurality of disposable lancets having a lancet tip for piercing a user's skin are provided on a carrier tape that is or can be wound onto a spool and used for obtaining a sample of body fluid. The lancets can each be arranged in a package and the lancet packages formed in this manner can be applied individually to the carrier tape and fastened thereon.

### CROSS-REFERENCES TO RELATED APPLICATIONS

This application is a continuation of International Patent Application No. PCT/EP2008/055689, filed May 8, 2008, which claims priority to European Patent Application No. 07108252.3, filed May 15, 2007.

### FIELD OF THE INVENTION

The invention concerns a method for storing lancets and optionally test elements in particular, for blood glucose analyses in which a plurality of disposable lancets provided with a lancet tip for piercing the skin are provided on a carrier tape which is wound onto or can be wound onto a spool for obtaining a sample of body fluid. The invention additionally concerns a tape magazine manufactured by such a method.

### DESCRIPTION OF RELATED ART

An arrangement of lancets on a carrier tape is known from International Patent Application WO-A 2005/107596 where the individual lancets can be successively brought into use and also disposed of again by tape transport. The lancets should be integrated on the tape by a direct connection in the course of the manufacturing process. In addition an additional provision of a test medium on the tape is also disclosed. This can simplify the handling for the user when carrying out self tests especially for blood sugar monitoring.

Based on this, the inventors desired to further improve the manufacturing methods and products of the methods known in the prior art and to design them such that a low-priced mass production with high product quality is possible.

### BRIEF SUMMARY OF THE INVENTION

It is against the above background that the present invention provides certain unobvious advantages and advancements over the prior art. In particular, the inventors have recognized a need for improvements in manufacturing methods for carrier tapes comprising lancets and in tape magazines for storing lancets.

Although the present invention is not limited to specific advantages or functionality, it is noted that the present invention is based on the idea of packaging the disposable elements separately on a first carrier and then to apply them like a label on a second carrier. Accordingly it is proposed according to the invention that the lancets are arranged each in a package and are thereby tightly enclosed in a sterile manner at least in the area of their tip which penetrates into the skin, and that subsequently the lancet packages formed in this manner are separated and applied individually to the carrier tape and attached thereon. This results in special advantages for storing lancing elements that have to be kept sterile. The prepackaging allows a sterile protection even in a manufacturing environment and at the same time simplifies the mechanical handling when they are joined to the carrier tape. Moreover, the packaging also results in an increased storage stability.

In order to create a flat structure that can be rolled up, it is advantageous when the packages consist of a base foil and a cover foil that is joined thereto. The base foil and cover foil can be brought into contact with one another and joined while enclosing the lancets.

Additional components can be avoided due to the fact that the base foil and the cover foil are joined together by a hot-melt adhesive join wherein a layer of hot-melt adhesive which is preferably on the base foil is activated by a hot stamp.

There are advantages for the manufacturing due to the fact that a plurality of lancet packages are manufactured in a connected manner as a package tape and are separated before application to the carrier tape. Another simplification of the manufacturing process provides that the package tape is formed by bringing together and joining a base foil strip and a cover foil strip. Alternatively it is also possible that the package tape is manufactured by folding down a foil strip and forming lancet pockets in the folded foil strip.

A sterile packaging can be achieved in that the packages are at the same time cut and the border sealed while enclosing the lancets by means of a hot cut.

In order to simplify the device handling it is advantageous when the lancets are permanently joined to the package, preferably at a proximal end section of the said lancets, and are preferably joined to the base foil.

A particularly preferred embodiment provides that the lancets and at least the tip area thereof are protected against damage and/or are kept sterile in the package.

The lancet tips are advantageously arranged in a pocket formed by the package. It is also conceivable that the packages are formed by a base foil and a biocompatible sealing compound applied thereon and especially consisting of silicone or TAE which encloses at least the lancet tip. In any case the pocket or the sealing block should be easy to penetrate in order to release the lancet tip for use.

Another advantageous embodiment is that at least the tips of the lancets in the lancet packages are sterilized preferably by high-energy irradiation before they are applied to the carrier tape. This enables a large number of them to have a targeted sterile protection as well as enabling the lancet packages to be subsequently handled outside of a sterile zone.

The application onto the carrier tape can be simply realized by fastening each of the lancet packages on the carrier tape at a joining surface that faces away from the lancet. The lancet packages are particularly preferably applied to the carrier tape in the form of labels or stickers. In this case it is advantageous when the lancet packages are provided with a piece of adhesive tape to glue them onto the carrier tape.

Basically it is also possible that the lancet packages are held on the carrier tape by mechanical connecting means or by welding (e.g. by ultrasound or laser).

A particularly preferred variant of the invention provides that test elements to which a sample of body fluid can be applied are applied to the carrier tape preferably alternating with the lancet packages. Accordingly it is proposed that the lancets and the test elements are prefabricated separately as package units and that subsequently the package units are applied to and fastened on the carrier tape in a common assembly process. In this manner it is possible to uniformly handle completely different disposable units when they are applied to the tape. In this connection the similar prepackaging which, however, is carried out separately enables different requirements with regard to the sterile protection of the lancets and the protection of the test chemistry to be especially taken into consideration.

The manufacturing is further simplified due to the fact that the flat lancet and test element package units have an essentially corresponding width which preferably corresponds to the width of the carrier tape.

Further handling advantages can also be achieved due to the fact that the carrier tape is arranged in a cassette such that it can be pulled off a take-off spool and wound onto a take-up spool.

With regard to a tape magazine as a product, a carrier tape that can be or is wound onto a spool and a plurality of disposable lancets arranged on the carrier tape are provided wherein the lancets are arranged each in a prefabricated or separate package and wherein the lancet packages are fastened on the carrier tape spaced apart from one another.

The invention also concerns a tape magazine for storing lancets and test elements especially for blood glucose analyses wherein the lancets and test elements are arranged each in a prefabricated package unit and wherein the lancet and test element package units are fastened on the carrier tape preferably in an alternating manner at a distance from one another.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE SEVERAL VIEWS OF THE DRAWING

The following detailed description of the embodiments of the present invention can be best understood when read in conjunction with the following drawings, where like structure is indicated with like reference numerals and in which:

FIG. 1 shows a lancet tape in an interrupted perspective view.

FIG. 2 shows an enlargement of a section of the lancet tape according to FIG. 1.

FIG. 3 shows the layer structure of the lancet tape in an exploded, sectional diagram.

FIGS. 4 and 5 show a section through the lancet tape longitudinally to the lancet and in the transverse direction to the lancet, respectively.

FIGS. 6 and 7 show a diagram of various steps for manufacturing lancet packages.

FIG. 8 shows a stamp for heat sealing lancet packages in a top-view.

FIG. 9 shows a section taken along the line 9-9 of FIG. 8.

FIG. 10 shows a tape cassette with a test tape furnished with lancets and test elements in a partially cut-open perspective view.

FIGS. 11 and 12 show a lancet being put into use in a perspective and sectional view, respectively.

Skilled artisans will appreciate that elements in the figures are illustrated for simplicity and clarity and have not necessarily been drawn to scale. For example, the dimensions of some of the elements in the figures may be exaggerated relative to other elements to help improve understanding of the embodiments of the present invention.

### DETAILED DESCRIPTION OF THE INVENTION

For the purposes of describing and defining the present invention it is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

The lancet supply tapes 10 shown in the drawing enable storage or provision of a plurality of lancets 12 as single-use articles for blood glucose determinations or other analyses in which a sample of body fluid is to be obtained for diagnostic purposes by a skin puncture. In addition, combinations of lancets and diagnostic test elements are also possible on a common supply tape. In both cases, prefabricated package or carrier units are prepared which are stored on a carrier tape in the form of a tape spool.

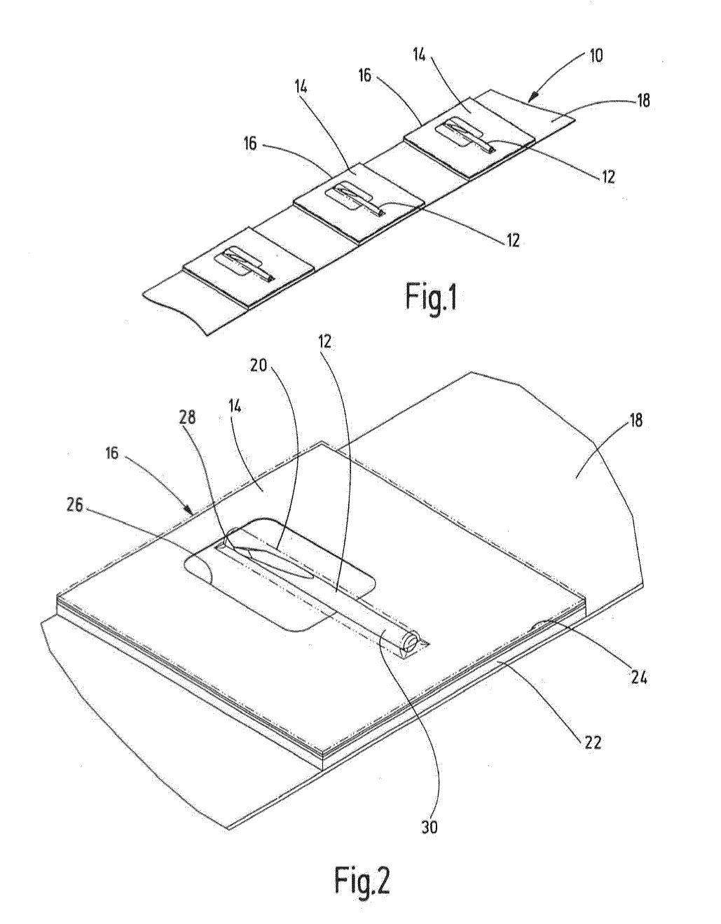

As shown in FIG. 1, the lancets 12 are each arranged in an allocated package 14 wherein the package units or lancet packages 16 formed in this manner are fastened on the carrier tape 18 like labels at a distance from one another. Since the lancet packages 16 are flexible and flat, this results in a tape magazine that can be rolled up and can be used in a hand-held device that is not shown for automatic handling.

In the enlargement of FIG. 2 it can be seen that the respective lancet 12 is protected in a pocket 20 formed by the package 14. The pocket 20 is formed by a foil laminate consisting of a base foil 22 and a cover foil 24. The lancet tip 28 is accommodated in an enlarged pocket area 26 in which it lies freely whereas the proximal shaft section 30 of the lancet 12 is tightly enclosed. Mechanical handling is thus facilitated even in the case of very small needle elements without having to be afraid that the very sensitive tip will be damaged or its sterility impaired. In the embodiment shown a round lancet aligned in the tape transverse direction is provided. Other orientations and designs are also conceivable for example in the form of a flat lancing element provided with a groove-shaped capillary collecting channel.

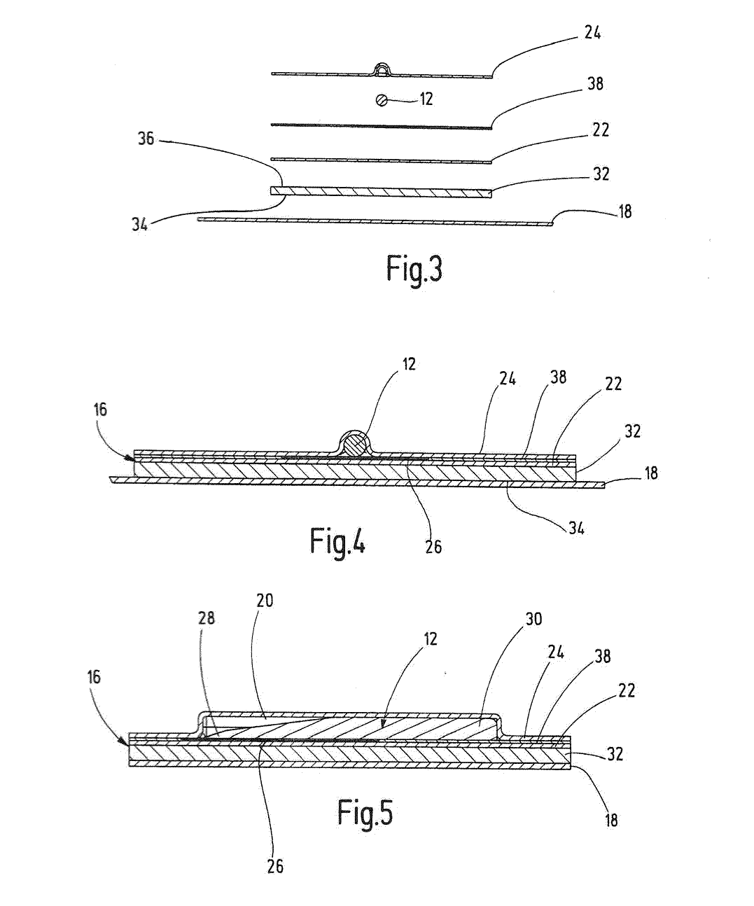

FIG. 3 shows the individual components of the foil structure according to FIG. 2. An approximately 10 μm thick and about 5-10 mm wide PET foil can for example be used as the carrier tape 18. A section of a double-sided adhesive tape 32 which has a first adhesive face 34 for the carrier tape 18 and an opposing second adhesive face 36 for the base foil 22 mediates the joining of the label-like lancet package 16. Alternatively it is also possible to use a piece of adhesive tape which adheres on one side and at the same time forms the base foil. Instead of an adhesive bond an integral bond or mechanical connection (e.g. a Velcro fastener) are also conceivable. The base foil 22 and cover foil 24 are laminated with inclusion of the lancet 12 by means of a layer of hot-melt adhesive 38 on the base foil which is activated by a hot stamp as elucidated in the following.

FIGS. 4 and 5 show the final product according to FIG. 2 in a section at right angles and longitudinal to the lancet 12. The thin cover foil nestles over the lancet cross-section thus making a seal around the lancet 12. In this connection it can also be seen that the proximal section of the lancet 30 is fastened on the base foil 22 by means of the layer of hot-melt adhesive 38 whereas the distal section of the lancet 28 is kept free for use in the enlarged unwelded area 26 of the pocket 20. Especially in the transverse orientation of the lancet 12 that is shown at right angles to the longitudinal direction of the tape, it is advantageous when the lancet packages 16 have dimensions that correspond to the width of the carrier tape 18 taking into account manufacturing tolerances.

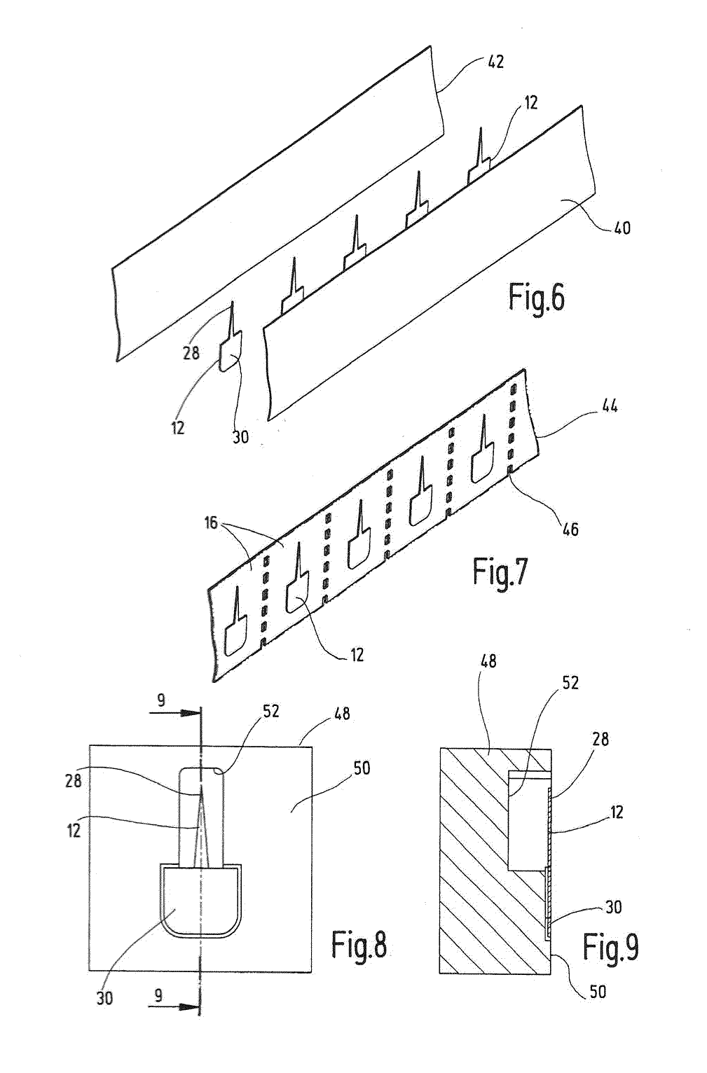

FIG. 6 illustrates the prefabrication of a package tape comprising a plurality of connected lancet packages. For this purpose a base foil strip 40 and a cover foil strip 42 are brought together and permanently joined to one another with enclosure of the lancets, which are in this case flat lancets 12 with a lancet tip 28 and a planar proximal holding member 30. An approximately 20 μm thick PET foil can for example be used as a base foil strip 40 which is coated on one side with a hot-melt adhesive made of a low-melting polyester, e.g., an ethylene vinyl acetate copolymer. The cover foil strip 42 is advantageously configured to be thinner than the base foil strip 40 and is for example a PET foil of 5 μm thickness.

As shown in FIG. 7, the package tape 44 can be provided with transverse perforations 46 which simplify the separation of the lancet packages 16 before they are applied to the carrier tape 18. It is also possible to divide and at the same time weld individual packages along the cutting line by a hot-cutting means in which case thermoplastic foil material is used. Another variant provides that a foil strip that is longitudinally folded is fitted with lancets and is transversely divided into individual packages (not shown). In this case one longitudinal half of the foil strip forms the base region for applying the lancets whereas the other longitudinal half is subsequently folded over and joined to the base region.

FIGS. 8 and 9 show the scaling of the individual lancet packages by a hot stamp 48 which acts on the hot-melt adhesive. The stamp face 50 of this stamp that presses against the respective lancet package 16 is provided with a stepped recess 52 which tightly encloses the proximal needle section 30 and due to a corresponding depression provides the enlarged unwelded region 26 of the pocket 20 in the area of the needle tip 28. In this case the proximal needle section 30 is preferably permanently glued to the base foil 22 such that the needle 12 can be re-stored in connection with the carrier tape 18 after a later use.

Although not shown specifically the lancets 12 sealed into the lancet packages 16 can be sterilized by irradiation for example with a high-energy electron beam. The material-tight seal ensures that it keeps sterile even in the subsequent processing sequence.

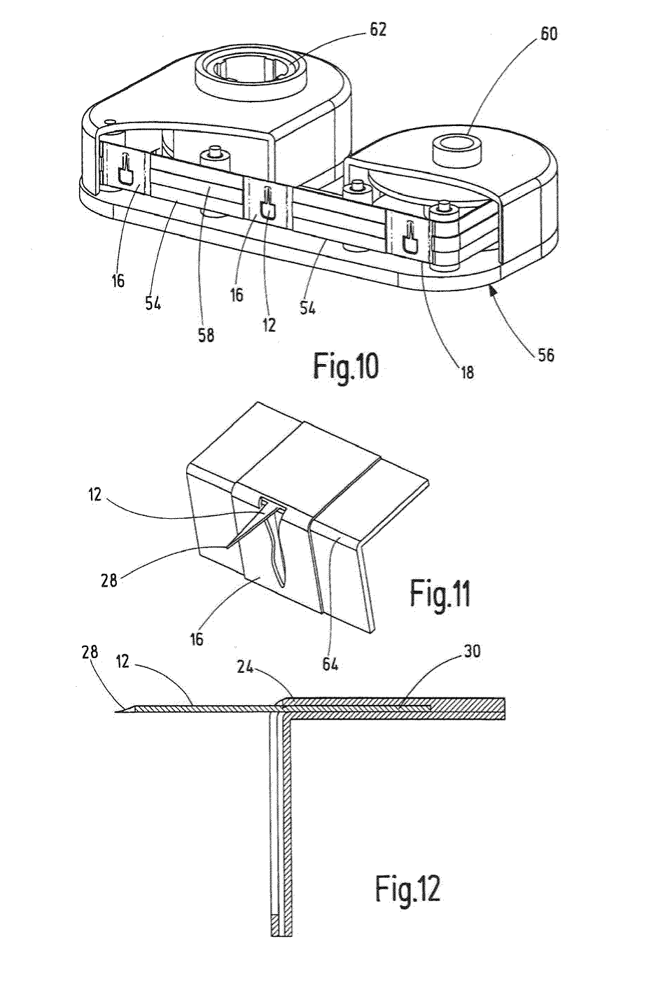

The lancet packages 16 are stuck onto the carrier tape 18 at a labeling station. In this connection it is also possible to apply additional test elements in the form of test field labels 54 as shown in FIG. 10 with the tape cassette 56 as an example. The test field labels 54 are provided with a reagent layer 58 to which body fluid (blood and/or tissue fluid) obtained by a skin puncture can be applied in order to detect an analyte (e.g., glucose). The analyte is preferably detected by means of a color change but it can also be detected electrochemically. The mechanical handling of the various disposable units 16, 54 at the labeling station can thus be carried out in a common labeling process as described in European Patent Application No. 1 593 434 A only for test field labels to which reference is herewith made.

The cassette 56 has a supply spool 60 for unused tape material and a take-up spool 62 for re-storing or disposing of used units 16, 54. They are provided by successively advancing the tape preferably in a handheld device in order to enable a substantially automatic measuring process.

In the course of such a measurement which can be carried out locally by the patient himself; the respective lancet 12 according to FIGS. 11 and 12 is brought into use. The thin cover foil 24 is slit open by the lancet 12 by bending the lancet package 16 at a deflection point 64 thus exposing the lancet tip 28. A lancing movement can then be carried out by a suitable actuator in order to for example prick a finger. The proximal lancet section 30 advantageously remains joined to the foil laminate in this process in order to simplify the subsequent disposal of the lancet 12 on the carrier tape 18.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

1. A method for manufacturing a carrier tape, adapted to be wound onto a spool, the carrier tape having a plurality of disposable lancets provided thereon, the lancets each having a lancet tip to pierce a user's skin and obtain a sample of body fluid, comprising:
providing sterilized lancets and arranging each lancet in a package so that the lancets are thereby sterilely enclosed at least in the area of their tips before they are applied to the carrier tape; and
subsequently individually applying the lancet packages formed in this manner to the carrier tape and attaching them thereon.

2. The method of claim 1 wherein the packages are formed from a base foil and a cover foil that is joined thereto.

3. The method of claim 2 wherein the base foil and cover foil are brought into contact with one another and joined while enclosing the lancets.

4. The method of claim 2 wherein the base foil and the cover foil are joined together by a hot-melt adhesive activated by a hot stamp.

5. The method of claim 1 wherein a plurality of the lancet packages are manufactured in a connected manner as a package tape and are separated before application to the carrier tape.

6. The method of claim 5 wherein the package tape is formed by bringing together and joining a base foil strip and a cover foil strip.

7. The method of claim 5 wherein the package tape is manufactured by folding down a foil strip and forming lancet pockets in the folded foil strip.

8. The method of claim 1 wherein the lancet packages are at the same time cut and the border is sealed while enclosing the lancets by using a hot cut.

9. The method of claim 1 wherein the lancets are permanently joined to the packages at a proximal end section of the lancets.

10. The method of claim 1 wherein at least the tips of the lancets are protected against damage in the packages.

11. The method of claim 1 wherein the lancet tips are arranged in pockets formed by the packages.

12. The method of claim 1 wherein the lancet packages are formed by a base foil and a sealing compound applied thereon which encloses at least the lancet tips.

13. The method of claim 1 wherein at least the tips of the lancets in the lancet packages are sterilized before they are applied to the carrier tape.

14. The method of claim 1 wherein the lancet packages are each fastened on the carrier tape at a joining surface that faces away from the lancet.

15. The method of claim 1 wherein the lancet packages are applied to the carrier tape in the form of labels or stickers.

16. The method of claim 1 wherein the lancet packages are provided with pieces of adhesive tape to glue them onto the carrier tape.

17. The method of claim 1 wherein the lancet packages are held on the carrier tape by mechanical connecting means or by welding.

18. The method of claim 1 wherein test elements to which the sample of body fluid can be applied are applied to the carrier tape.

19. The method of claim 18 wherein the test elements are applied in alternating fashion on the carrier tape with the lancet packages.

20. A method for manufacturing a carrier tape adapted to be wound onto a spool, the carrier tape having a plurality of lancets and test elements provided thereon, the lancets being configured for piercing a user's skin, and the test elements being configured for analyzing a sample of body fluid, comprising:
prefabricating the lancets and the test elements separately as package units; and
subsequently applying the package units to the carrier tape and fastening them on the carrier tape in a common assembly process.

21. The method of claim 20 wherein the lancet and test element package units have widths which correspond to the width of the carrier tape.

22. The method of claim 20 wherein the carrier tape is arranged in a cassette so that it can be pulled off a take-off spool and wound onto a take-up spool.

23. A tape magazine for storing lancets, the magazine comprising a carrier tape wound onto a spool and a plurality of disposable lancets arranged on the carrier tape, the lancets each comprising a lancet tip for piercing a user's skin, wherein the lancets are each arranged in a prefabricated package and the lancet packages are fastened on the carrier tape in spaced apart relationship from one another.

24. A tape magazine for storing lancets and analyte test elements, the magazine comprising a carrier tape wound onto a spool and a plurality of disposable lancets and analyte test elements arranged on the carrier tape, wherein the lancets and test elements are each arranged in a prefabricated package unit and the lancet and test element package units are fastened on the carrier tape in an alternating manner spaced apart from one another.
